(19) United States
(12) Patent Application Publication
(10) Pub. No.: US 2024/0189130 A1
(43) Pub. Date: Jun. 13, 2024

(54) STAPLER FOR PERFORMING LAPAROSCOPIC GASTRIC SLEEVE PROCEDURES

(71) Applicant: KING FAISAL UNIVERSITY, AL-AHSA (SA)

(72) Inventor: ABDULRAHMAN SALEH ALMULHIM, AL-AHSA (SA)

(21) Appl. No.: 18/079,838

(22) Filed: Dec. 12, 2022

(51) Int. Cl. A61F 5/00; A61B 17/072

(52) U.S. Cl. CPC A61F 5/0089; A61B 17/072; A61B 2017/07271

(57) ABSTRACT

The stapler for performing laparoscopic gastric sleeve procedures is similar to a conventional surgical stapler, including a handle assembly, a shaft extending distally from a distal end of the handle assembly, and an end effector mounted on a distal end of the shaft. The end effector includes a lower jaw and an upper jaw, the lower jaw being adapted for supporting a staple cartridge and the upper jaw having an anvil surface facing the lower jaw. A proximal end of at least one of the lower and upper jaws is convexly curved, thus forming an at least substantially semicircular or semielliptical open space between the proximal ends of the lower and upper jaws, enabling a bypass sleeve to be formed by stapling without resection of the stomach.

### BACKGROUND

### 1. Field

The disclosure of the present patent application relates to bariatric surgery, and particularly to a surgical stapler for performing laparoscopic gastric sleeve procedures.

### 2. Description of the Related Art

Laparoscopic sleeve gastrectomy (LSG) is a form of bariatric surgery offered to patients who are morbidly obese (having a high body mass index) as an alternative to gastric bypass surgery, or sometimes as the first stage in a treatment plan followed by gastric bypass surgery (or other bariatric procedure) in the second stage, the object being surgical weight loss. LSG is basically a minimally invasive procedure in which the fundus and a major portion of the body of the stomach is resected and removed. The portion of the stomach that remains is a banana-shaped tube directly connected to the small intestine that resembles a sleeve. Since there is less stomach for receiving food, the patient feels full after eating smaller portions, and very often the portion of the stomach that produces the hormone ghrelin (a hormone that signals or stimulates feelings of hunger) is removed during the procedure. The result is a significant weight loss that is maintained if the patient maintains the recommended dietary change to smaller, though more frequent, portions of food at meals and foregoing eating between meals.

LSG is typically viewed as a faster and simpler procedure than its alternatives. In order to discuss the LSG procedure in more detail, reference is first made to FIG. 2, which illustrates a typical human digestive system 100. Food enters the esophagus 180 and then passes into the body 184 of the stomach 110. In the example of FIG. 2, the body 184 of the stomach 100 includes an excess portion 182. In order to decrease the available volume of stomach 100, this excess portion 182 will be resected using LSG. Once the food is digested in the stomach 100, it enters the pylorus 186 (the opening between the stomach and the small intestine) and continues through the duodenum (the first part of the small intestine 188). The lesser curvature 194 is the portion of the stomach 100 that will remain after LSG. The greater curvature 192 is an area opposite the opening into the stomach where tissue is typically removed during LSG. During LSG, surgeons generally trim and/or staple substantially parallel to the greater curvature 192 and/or the lesser curvature 194. The lesser sac (not shown) is an area generally behind the body 184 of the stomach 100, and typically may be freed prior to the trimming along the greater curvature 192.

As shown in FIG. 3, during LSG, a balloon device 120 may be introduced into the stomach 100 through the esophagus 180, extending into the region of the lesser curvature 194. The balloon device 120, once inflated, maintains the shape and position of the portion of the stomach that will be retained. The stomach 100 is divided into the retained portion 130, which has a tube or sleeve shape, and a resected portion 140, which is removed. The surgeon trims and/or staples the excess portion 140 to be dissected using a surgical stapler 150. Surgical staplers for performing LSG typically include a cutting blade with the stapling head, allowing the surgical stapler 150 to cut, divide, separate, and/or remove the excess portion 140 from the body 184 of the stomach 100. As shown in FIG. 3, as the surgeon dissects the excess portion 140, surgical staples 160 are simultaneously stapled by the stapling head of surgical stapler 150 to close the tissue.

Removal of the resected portion 140 typically reduces the body's capacity to produce the hormone ghrelin, which may decrease stimulation of hunger in the patient. Further, the pylorus 186 is generally not removed during LSG, allowing food to remain in the stomach longer in order to enhance the feeling of fullness until the food is ultimately passed on.

Surgical staplers for performing LSG are typically similar to the cutting surgical staplers that are commonly in use for a wide variety of purposes. An example of such cutting surgical staplers is shown in U.S. Pat. No. 10,194,936, issued Feb. 5, 2019 to Vendely et al., which is hereby incorporated by reference in its entirety. As shown in FIGS. 4-6, an exemplary conventional surgical stapler 150 for performing LSG includes a handle assembly 152, a shaft 154 extending distally from an end 156 of the handle assembly 152, and an end effector 158 mounted at an end 162 of the shaft 154 opposite the handle assembly 152. The end effector 158 has opposed linear lower and upper jaws 164, 166, the lower jaw 164 having a staple channel 168 disposed therein configured to support a staple cartridge 170. The upper jaw 166 has an anvil surface 172 that faces the lower jaw 164 and is configured to operate as an anvil to help deploy staples from the staple cartridge 170.

At least one of the opposed lower and upper jaws 164, 166 is movable relative to the other jaw to clamp tissue and/or other objects disposed therebetween. Components of a firing system can be configured to pass through at least a portion of the end effector 158 to eject the staples into the clamped tissue. A knife blade 174 (shown in FIG. 6) or other cutting element can be associated with the firing system to cut tissue during the stapling procedure.

The handle assembly 152 is used by the surgeon to operate the end effector 158. In the exemplary stapler 150 of FIG. 4, the handle assembly 152 has a pistol-grip type housing 176 with a variety of mechanical and/or electrical components disposed therein to operate various features of the stapler 150. For example, the handle assembly 152 can include a rotation knob 178 mounted adjacent the distal end 156 for facilitating rotation of the shaft 154 and/or the end effector 158 with respect to the handle assembly 152 about a longitudinal axis L of the shaft 154. The handle assembly 152 can further include clamping components as part of a clamping system actuated by a clamping trigger 102 and firing components as part of the firing system that are actuated by a firing trigger 104. The clamping and firing triggers 102, 104 can be biased to an open position with respect to a stationary handle 106 by a torsion spring, for example. Movement of the clamping trigger 102 toward the stationary handle 106 can actuate the clamping system, which causes the jaws 164, 166 to collapse towards each other, thus clamping tissue therebetween. Movement of the firing trigger 104 can actuate the firing system, which causes the ejection of staples from the staple cartridge 170 disposed therein and/or the advancement the knife blade 174 to sever tissue captured between the jaws 164, 166.

In the exemplary stapler 150 of FIG. 5, the staple cartridge 170 is supported in a staple tray 108, which is supported within the cartridge channel 168 of the lower jaw 164. The upper jaw 166 has a plurality of staple forming pockets, each of which is positioned above a corresponding staple from the plurality of staples contained within the staple cartridge 170. The upper jaw 166 can be connected to the lower jaw 164 in a variety of ways, although in the stapler 150 of FIG. 5, the upper jaw 166 has a proximal pivoting end 112 that is pivotally mounted within a proximal end 114 of the staple channel 168, just distal to its engagement to the shaft 154. When the upper jaw 166 is pivoted downward, the upper jaw 166 moves the anvil surface 172 and the staple-forming pockets formed thereon move toward the opposing staple cartridge 170.

Various clamping components can be used to effect opening and closing of the jaws 164, 166 to selectively clamp tissue therebetween. In the exemplary stapler 150 of FIG. 5, the pivoting end 112 of the upper jaw 166 includes a closure feature 116 distal to its pivotal attachment with the staple channel 168. A closure tube 118, whose distal end includes an aperture 122 that engages the closure feature 116, selectively imparts an opening motion to the upper jaw 166 during proximal longitudinal motion and a closing motion to the upper jaw 166 during distal longitudinal motion of the closure tube 118 in response to the clamping trigger 102.

The firing components of the exemplary stapler 150 of FIGS. 5 and 6 include a firing bar 124 having an E-beam 126 on a distal end thereof. The firing bar 124 is encompassed within the shaft 154, for example, in a longitudinal firing bar slot 128 of the shaft 154, and guided by a firing motion from the handle 152. Actuation of the firing trigger 104 effects distal motion of the E-beam 126 through at least a portion of the end effector 158 to cause the firing of staples contained within the staple cartridge 170. In the exemplary stapler 150 of FIGS. 5 and 6, guides projecting from a distal end of the E-Beam 126 engage a wedge sled 134, which, in turn, pushes staple drivers 136 upward through staple cavities 138 formed in the staple cartridge 170. Upward movement of the staple drivers 136 applies an upward force on each of the plurality of staples within the cartridge 170 to thereby push the staples upward against the anvil surface 172 of the upper jaw 166 and create formed staples.

Since, as shown in FIG. 3, the dissection begins at the lower end of the stomach 100 and proceeds all the way upward, the entire vertical length must be cut. Since the amount of potential bleeding and leakage is directly proportional to the length or surface area being cut, it would be desirable to minimize the extent of cutting, or even opening, of the tissue in order to reduce the possibility of complications to the patient. Thus, a stapler for performing laparoscopic gastric sleeve procedures solving the aforementioned problems is desired.

### SUMMARY

The stapler for performing laparoscopic gastric sleeve procedures includes a handle assembly, a shaft extending from an end of the handle assembly, and an end effector mounted on an end of the shaft opposite the handle assembly. The end effector includes a lower jaw and an upper jaw, the lower jaw being adapted for supporting a staple cartridge and the upper jaw having an anvil surface facing the lower jaw. Each of the lower and upper jaws is convexly curved adjacent the jaw end of the shaft, thus forming a substantially circular or elliptical open space between the ends of the lower and upper jaws and the shaft. Alternatively, only one of the lower and upper jaws may be provided with a convexly curved proximal end, with the other one of the jaws being substantially straight.

The stapler for performing laparoscopic gastric sleeve procedures is used in a manner similar to a conventional surgical stapler, including the selective opening and closing of the lower and upper jaws with respect to one another and the selective firing of staples from the staple cartridge. However, the substantially circular or elliptical open space formed between the ends of the lower and upper jaws attached to the shaft allows the distal portion of the stomach to be held therein such that the lower and upper jaws may be closed without damaging the distal portion of the stomach. The result is that the laparoscopic sleeve is separated from the fundus and body of the stomach by stapling, without resection of the fundus or body of the stomach.

In use, the abdomen is inflated, such as with a balloon device, a 5 mm trocar is placed at or above the patient's umbilicus (depending on the size of the patient), two 5 mm trocars are placed on both sides of the abdomen as work ports, and a 12 mm trocar is placed on the left side of the umbilicus. The end effector of the stapler for performing laparoscopic gastric sleeve procedures is introduced through the port formed to the left of the umbilicus (by the 12 mm trocar), the lower and upper jaws are opened, and the end effector is passed anterior and posterior to the stomach. The surgeon proceeds to position the end effector up to the angle of Hiss at the level of the hiatal orifice. The proximal ends of the lower and upper jaws are open at this point and the distal portion of the stomach is fully positioned within the substantially circular or elliptical open space. In this way, the end effector may be used to fire staples without requiring dissection. The lower and upper jaws are then closed to fire the staples. The abdomen can then be examined and closed. This results in a sleeve portion, which is separated from the fundus and lateral body of the stomach by a stapled region. Based on average adult sizes of stomachs, the stapled region may extend approximately 25 to 30 cm, and the intact distal portion of the stomach may have a remaining length of approximately 5 to 7 cm, resulting in formation of a sleeve without cutting or resection.

These and other features of the present subject matter will become readily apparent upon further review of the following specification and drawings.

### BRIEF DESCRIPTION OF THE DRAWINGS

FIG. 1 is a side view of a stapler for performing laparoscopic gastric sleeve procedures.

FIG. 2 is a schematic diagram showing a portion of a typical human digestive system.

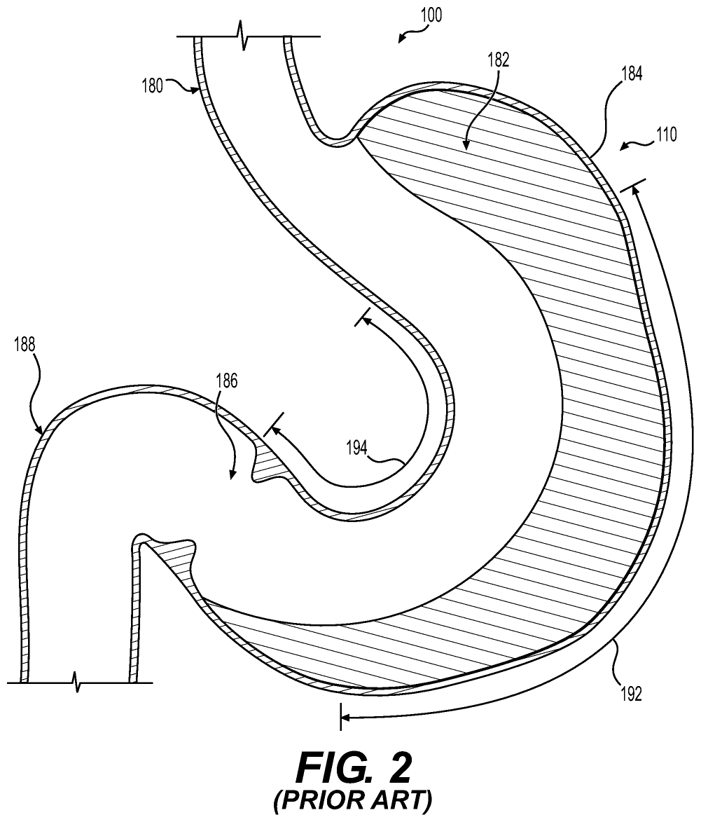

FIG. 3 is a schematic diagram showing cutting and stapling during a typical laparoscopic sleeve gastrectomy procedure.

FIG. 4 is a perspective view of an exemplary surgical stapler of the prior art.

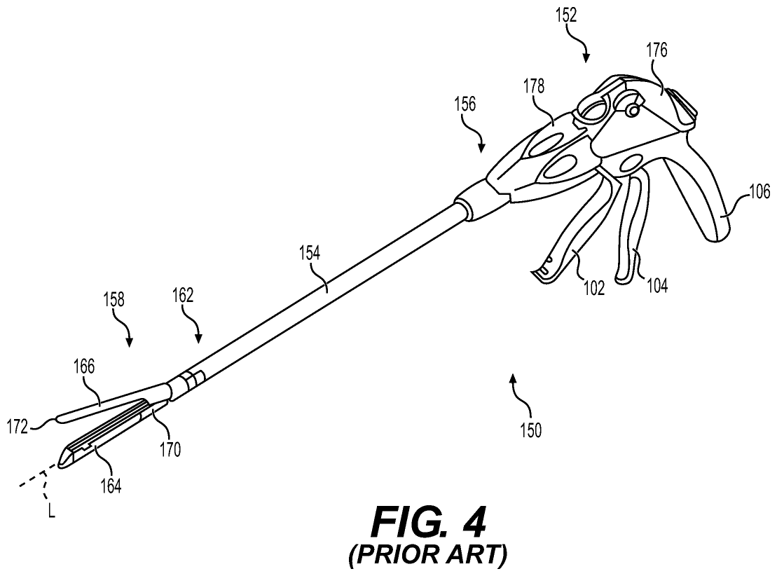

FIG. 5 is an exploded perspective view of an end effector of the prior art surgical stapler of FIG. 4.

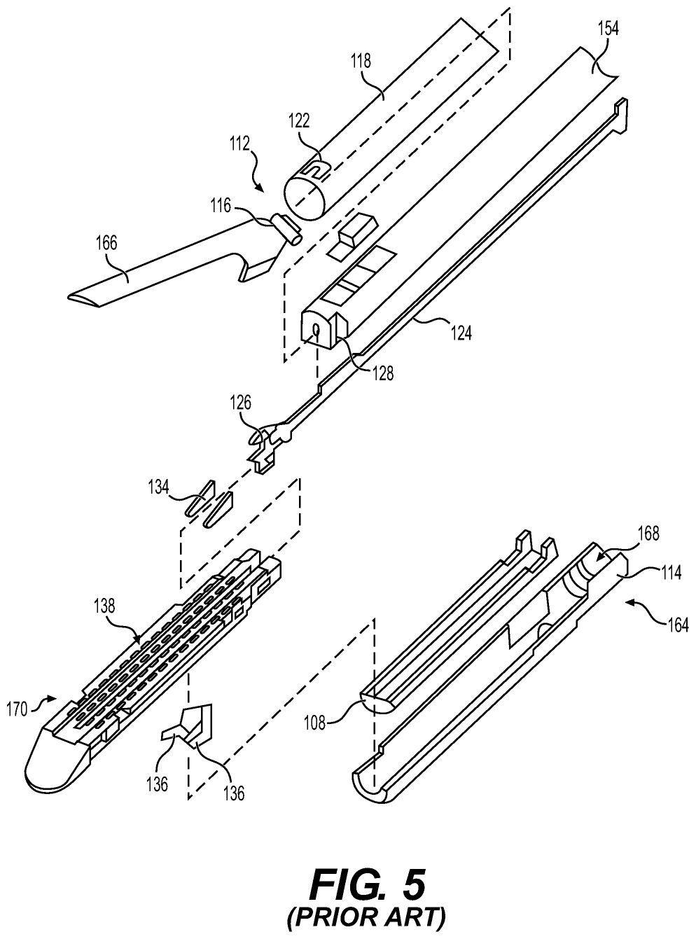

FIG. 6 is a perspective view of a firing bar of the prior art surgical stapler of FIG. 4.

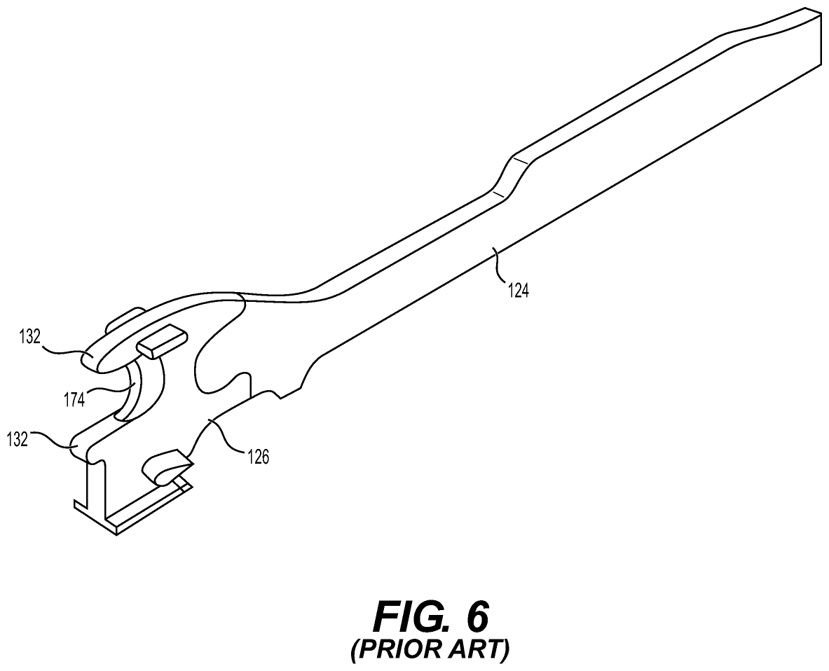

FIG. 7A illustrates the stapler of FIG. 1 in use while performing a laparoscopic gastric sleeve procedure.

FIG. 7B illustrates formation of a gastric sleeve following the procedure of FIG. 7A.

FIG. 8 is a side view of an alternative embodiment of a stapler for performing laparoscopic gastric sleeve procedures in which the upper jaw is linear or straight throughout its length.

Similar reference characters denote corresponding features consistently throughout the attached drawings.

### DETAILED DESCRIPTION OF THE PREFERRED EMBODIMENTS

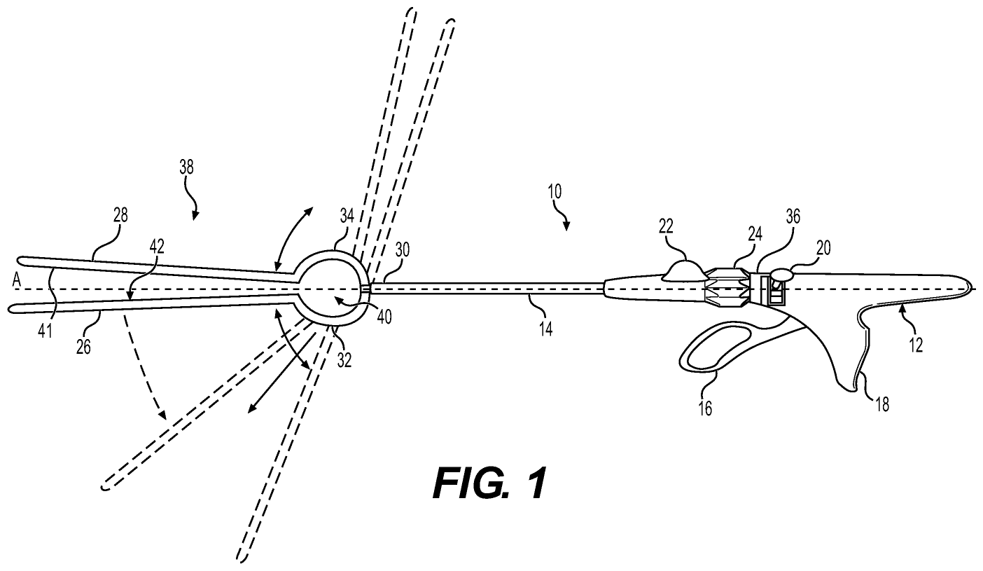

The stapler for performing laparoscopic gastric sleeve procedures 10 of FIG. 1 includes a handle assembly 12, a shaft 14 extending distally from a distal end 36 of the handle assembly 12, and an end effector 38 extending from a distal end 30 of the shaft 14. The handle assembly 12 may operate in a manner similar to the handle assembly 152 of the stapler 150, the shaft 14 may be similar to the shaft 154 of the stapler 150, and the end effector 38 may operate in a manner similar to the end effector 158 of the stapler 150. It should be understood that the overall dimensions and configuration of the handle assembly 12, shaft 14, and end effector 38 are shown in FIG. 1 for exemplary purposes only.

The end effector 38 includes a lower jaw 26 and an upper jaw 28. The lower jaw 26 is adapted for supporting a staple cartridge 42 and the upper jaw 28 has an anvil surface 41 facing the lower jaw 26. The stapler 10 is used in a manner similar to the stapler 150, and the selective opening and closing of the lower and upper jaws 26, 28 with respect to one another may be similar to that described above with regard to the opening and closing of the jaws 164, 166 of the stapler 150. Similarly, the selective firing of staples from the staple cartridge 42 supported in the lower jaw 26 may operate in a manner similar to that described above with regard to the staple cartridge 170 of the stapler 150. However, it should be understood that the lower jaw 26 and the upper jaw 28 may be selectively opened and closed with respect to one another in any suitable manner, as is well known in the art. Similarly, it should be understood that the staples in the staple cartridge 42 may be fired in any suitable manner, as is also well known in the art.

In FIG. 1, the handle assembly 12 is shown as having a pistol-grip type housing with a rotation knob 24 mounted adjacent the distal end 36 of the handle 12 for facilitating opening and closing of the end effector 38. It should be understood that the rotation knob 24 is shown for exemplary purposes only. Additionally, a second rotation knob 22 is provided, allowing the end effector 38 to be rotated with respect to the shaft 14 in a direction perpendicular to the longitudinal axis, i.e., in and out of the page in the orientation shown in FIG. 1. It should be understood that any suitable type of mechanical and/or electrical coupling with knobs 22, 24 may be used to effect the desired actions.

Further, in FIG. 1, a clamping trigger 20 is shown in the form of a switch, rather than the conventional clamping trigger 102 of the prior art stapler 150. It should be understood that the clamping trigger 102 may have any suitable configuration. A firing trigger 16 for firing the staples may be biased to an open position with respect to the stationary handle 18 by a resilient member, for example. Movement of the clamping trigger 20 actuates the clamping system, which causes the lower and upper jaws 26, 28 to collapse towards each other, thus clamping tissue therebetween. Movement of the firing trigger 16 actuates the firing system, which causes the ejection of staples from the staple cartridge 42 disposed therein.

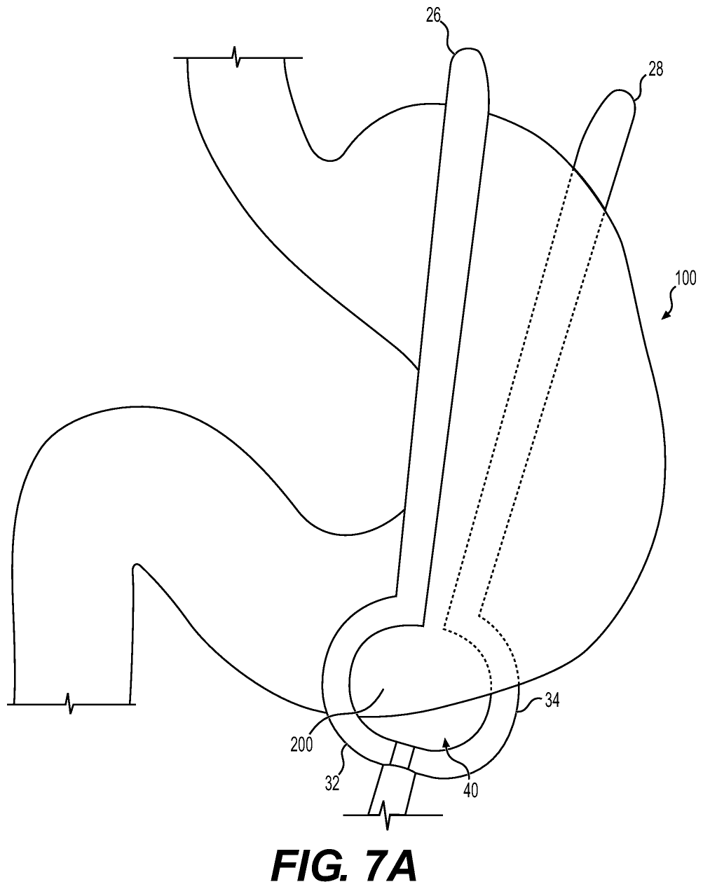

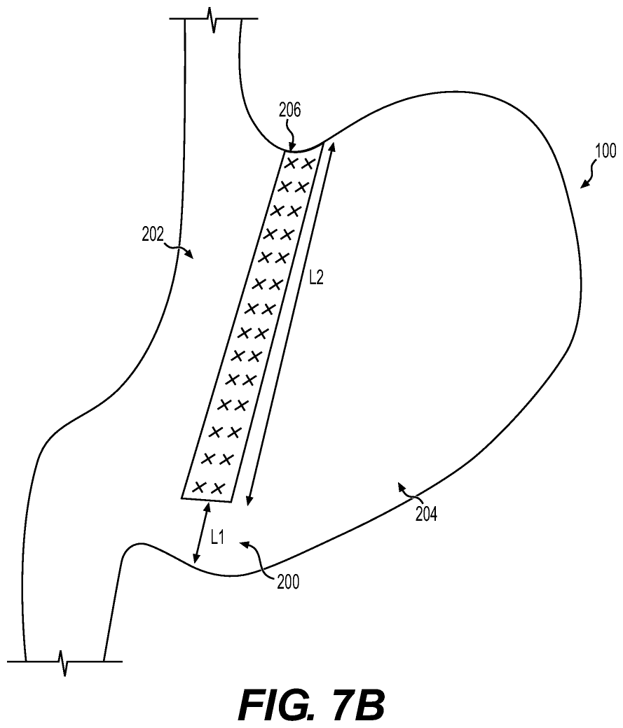

As further seen in FIG. 1, the proximal ends 32, 34 of the lower and upper jaws 26, 28, respectively, are each convexly curved, thus forming a substantially circular or elliptical open space 40 between the proximal ends 32, 34. The stapler 10 is used in a manner similar to a prior art surgical stapler, including the selective opening and closing of the lower and upper jaws 26, 28 with respect to one another and the selective firing of staples from the staple cartridge 42. However, as shown in FIG. 7A, the substantially circular or elliptical open space 40 formed between the proximal ends 32, 34 of the lower and upper jaws 26, 28, respectively, allows the distal portion 200 of the stomach 100 to be held therein such that the lower and upper jaws 26, 28 may be closed only above this region, without damaging or stapling the distal portion 200 of stomach 100. The laparoscopic sleeve procedure may then be performed primarily using stapling, without the typical resection of the greater curvature 192.

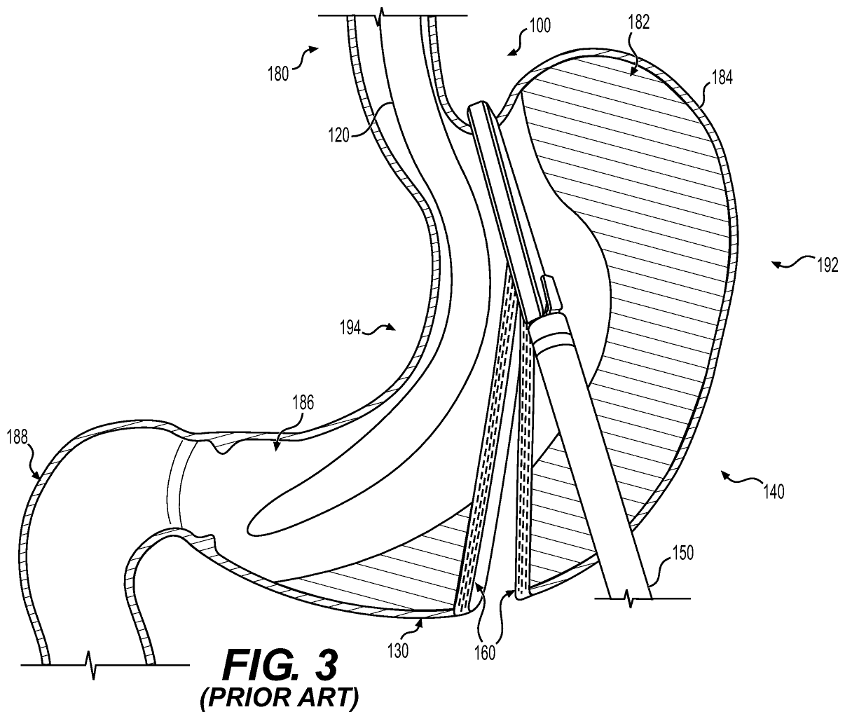

In use, the abdomen is inflated, such as with the balloon device 120 described above, a 5 mm trocar is placed at or above the patient's umbilicus (depending on the size of the patient), two 5 mm trocars are placed on both sides of the abdomen as work ports, and a 12 mm trocar is placed on the left side of the umbilicus. The end effector 38 of the stapler for performing laparoscopic gastric sleeve procedures 10 is introduced through the port formed to the left of the umbilicus (by the 12 mm trocar), the lower and upper jaws 26, 28 are opened, and the end effector 38 is passed anterior and posterior to the stomach 100. The surgeon proceeds to position the end effector 38 up to the angle of Hiss at the level of the hiatal orifice. The proximal ends 32, 34 of the lower and upper jaws 26, 28, respectively, are open at this point and the distal portion 200 of the stomach 100 is fully positioned within the substantially circular or elliptical open space 40 (as shown in FIG. 7A). In this way, the end effector 38 may be used to fire staples without requiring the dissection illustrated in FIG. 3, for example. The lower and upper jaws 26, 28 are then closed to fire the staples. The abdomen can then be examined and closed. The end result is shown in FIG. 7B, where a sleeve portion 202 is separated from the fundus and lateral body 204 by a stapled region 206. Based on average adult sizes of the stomach, the intact distal portion 200 of stomach 100 may have a remaining length L1 of approximately 5 to 7 cm, and the stapled region 206 may extend along a length L2 of approximately 25 to 30 cm, resulting in formation of a sleeve 202 without cutting or resection.

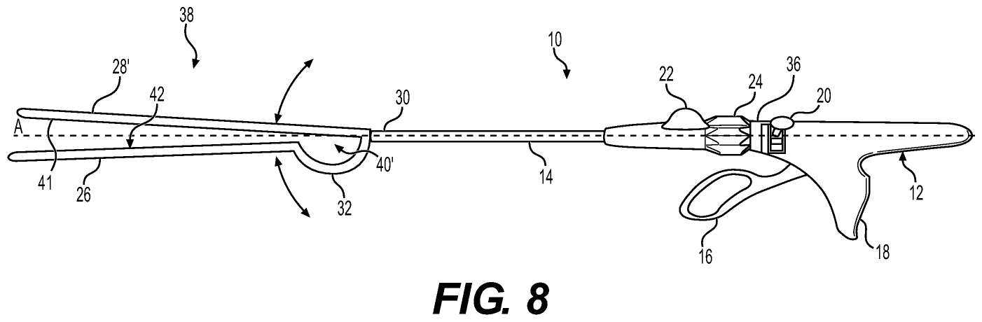

In order to maintain the intact distal portion 200, it is not required for each of the lower and upper jaws 26, 28 to have a convexly curved proximal end. In the alternative embodiment of FIG. 8, upper jaw 28 of FIG. 1 has been replaced by an upper jaw 28′, which is completely straight. Only the curved proximal end 32 of lower jaw 26 is required to form open space 40′. It should be understood that the lower jaw 26 could be replaced by a straight lower jaw, with the upper jaw 28 maintaining its curved proximal end.

It is to be understood that the stapler for performing laparoscopic gastric sleeve procedures is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A stapler for performing laparoscopic gastric sleeve procedures, comprising:
a handle assembly having a distal end;
a shaft extending distally from the distal end of the handle assembly, the shaft having a distal end;
an end effector mounted on the distal end of the shaft, the end effector having a lower jaw and an upper jaw, the lower jaw being adapted for supporting a staple cartridge and the upper jaw having an anvil surface facing the lower jaw, each of the lower and upper jaws having a convexly curved proximal end at the distal end of the shaft;
means for selectively opening and closing the upper and lower jaws with respect to one another; and
means for selectively deploying staples from the staple cartridge, wherein the end effector is rotatable with respect to the shaft such that the end effector is rotatable in a direction which is perpendicular to a longitudinal axis of said shaft, and wherein the longitudinal axis of said shaft is defined as an axis commencing at said distal end of said handle assembly and traversing the length of said shaft and terminating at said distal end of said shaft.

2. (canceled)

3. A stapler for performing laparoscopic gastric sleeve procedures, comprising:
a handle assembly having a distal end;
a shaft extending distally from the distal end of the handle assembly, the shaft having a distal end;
an end effector mounted on the distal end of the shaft, the end effector having a lower jaw and an upper jaw, the lower jaw being adapted for supporting a staple cartridge and the upper jaw having an anvil surface facing the lower jaw, at least one of the lower and upper jaws having a convexly curved proximal end at the distal end of the shaft;
means for selectively opening and closing the upper and lower jaws with respect to one another; and
means for selectively deploying staples from the staple cartridge, wherein the end effector is rotatable with respect to the shaft such that the end effector is rotatable in a direction which is perpendicular to a longitudinal axis of said shaft, and wherein the longitudinal axis of said shaft is defined as an axis commencing at said distal end of said handle assembly and traversing the length of said shaft and terminating at said distal end of said shaft.

4. (canceled)

5. The stapler for performing laparoscopic gastric sleeve procedures as recited in claim 3, wherein the lower jaw has the convexly curved proximal end at the distal end of the shaft.

6. The stapler for performing laparoscopic sleeve procedures as recited in claim 3, wherein the upper jaw has the convexly proximal end at the distal end of the shaft.

7. The stapler for performing laparoscopic gastric sleeve procedures as recited in claim 3, wherein both the upper and lower jaws have the convexly curved proximal end at the distal end of the shaft.
